(19) United States
(12) Patent Application Publication
(10) Pub. No.: US 2012/0058057 A1
(43) Pub. Date: Mar. 8, 2012

(54) Anti-dandruff Agents

(75) Inventors: William-Robert PITNER, Corinth, MS (US); Marlies WATERMANN, Buettelborn (DE); Jens EICHHORN, Reinheim (DE); Thomas RUDOLPH, Darmstadt (DE); Uschi SCHMID-GROSSMANN, Bensheim (DE)

(73) Assignee: MERCK PATENT GMBH, Darmstadt (DE)

(21) Appl. No.: 13/291,242

(22) Filed: Nov. 8, 2011

Related U.S. Application Data
(63) Continuation of application No. PCT/EP2010/002643, filed on Apr. 29, 2010.

(30) Foreign Application Priority Data
May 28, 2009 (EP) 09007113.5

(51) Int. Cl. A01N 37/44; A61K 8/368; A61K 8/44; A61Q 3/00; A61K 9/12; A01P 3/00; A61Q 17/00; C07C 229/22; A61Q 15/00

(52) U.S. Cl. 424/45; 562/567; 514/561; 424/59; 424/60; 424/65; 424/61

(57) ABSTRACT

The present invention relates to special ammonium carboxylates, their synthesis and application, particularly as anti-dandruff agents.

### CROSS-REFERENCE TO EARLIER FILED APPLICATIONS

The present application claims the benefit of and is a continuation of PCT International Application PCT/EP/2010/002643 filed Apr. 29, 2010, which claims the benefit of European Application EP 09007113.5 filed May 28, 2009, the entire disclosures of which are hereby incorporated by reference.

### FIELD OF THE INVENTION

The present invention relates to special ammonium carboxylates, their synthesis and their application as anti-dandruff agents.

### BACKGROUND OF THE INVENTION

Salts such as N,N,N-Trimethyl-2-hydroxyethylammonium butanoate are described as solvents for refined cork dissolution, particularly with respect to the separation of large quantities of suberin (H. Garcia et al, Green Chem., (2010), 12, 367-369).

Dandruff is a scalp disorder that is characterized by the formation of white or grey scales, accompanied by mild itching. The scales present diffusely and in patches. Dandruff occurs most frequently and most severely in young males, is rare in children and the elderly, and is otherwise common throughout the world's adult population. Dandruff has traditionally been linked to seborrhoea, an inflammatory skin disorder that is known for producing greasy scales superimposed upon reddened skin areas. However, seborrhoea can occur without dandruff, and dandruff can develop in the absence of apparent seborrhoea. Current knowledge suggests that the term “dandruff” is best used to describe the symptom complex of scalp flaking and itching, rather than as a synonym for seborrhoea, which is a specific disease entity. Although dandruff is a possible symptom of seborrhoea, it also can potentially result from scalp irritation caused by excessive sun exposure, airborne environmental substances, and cosmetic hair products. Dandruff reflects a fundamental abnormality in the dead outer layer of skin (“the scalp”) that covers the hairy top of the head. The involved skin cells lack the ability to properly adhere to one another. Consequently, clumps of cells separate from the scalp surface as scales. The shedding of these scales produces flakes of dandruff.

A relationship between dandruff and a class of yeast called Malassezia furfur and Malassezia globosa has long been recognized. Bacteria and yeast are ordinary occupants of the human scalp. However, in those individuals with dandruff, yeast is present in significantly greater numbers than would normally be expected. Many doctors and researchers believe that inflammation caused by an immune response to the yeast produces the dandruff condition.

Incorporation of anti-dandruff agents thus becomes essential in hair care products for the treatment of the infected scalp. However, many anti-dandruff compounds are not well seen as such in view of their long lasting protection against dandruff or their behavior in compositions. Therefore, there is a real need of effective anti-dandruff compounds which can be easily formulated in compositions like hair care formulations, preferably in the aqueous phase of said formulations.

### SUMMARY OF THE INVENTION

It is an object of the present invention to provide alternative anti-dandruff compounds. Surprisingly it has been found that a special class of ammonium carboxylates are especially useful as anti-dandruff agents. Therefore, the present invention is directed to compounds of formula I

[NRR²R²R³]⁺[R⁴—COO]⁻ I

in which

R is methoxyethyl, methoxymethyl, ethoxyethyl or ethoxymethyl,

R¹ to R³ are independantly of each other methyl or ethyl,

R⁴ is linear or branched alkyl with 2 to 4 C atoms.

Compounds of formula I can be seen as ionic liquids or liquid salts. Typically, ionic liquids are ionic species which consist of an organic cation and a generally inorganic or organic anion. They preferably do not comprise neutral molecules and preferably have melting points below 373 K. In the context of this patent application, the term “ionic liquid” is used as a synonym to chemical compound or compound.

Compounds of formula I have unique properties which means they are water soluble, thermally stable, readily biodegradable and they are effective anti-dandruff agents as documented in the examples.

The linear or branched alkyl group with 2 to 4 C-atoms is, for example, ethyl, n-propyl, isopropyl, n-butyl, sec-butyl or tert-butyl.

The subsitutent R⁴ in formula I is preferably linear alkyl with 2 to 4 C-atoms, particularly preferably ethyl or n-propyl, very particularly preferably ethyl.

The substituents R¹ to R³ in the compounds of the formula I may be identical or different. Preferably two substituents are identical and one substituent is different. Particularly preferably, two substituents of R¹ to R³ are methyl and the other substituent is ethyl or two substituents of R¹ to R³ are ethyl and the other substituent is methyl. Very particularly preferably, two substituents of R¹ to R³ are methyl and the other substituent is ethyl. The substituent R is preferably methoxyethyl or ethoxyethyl. Further preferred combinations are disclosed in the claims.

Preferred compounds of formula I are:

N-ethyl-N,N-dimethyl-2-methoxyethylammonium propionate,

N-ethyl-N,N-dimethyl-2-ethoxyethylammonium propionate,

N-ethyl-N,N-dimethyl-2-methoxymethylammonium propionate,

N-ethyl-N,N-dimethyl-2-ethoxymethylammonium propionate,

N-ethyl-N,N-dimethyl-2-methoxyethylammonium butanoate,

N-ethyl-N,N-dimethyl-2-ethoxyethylammonium butanoate,

N-ethyl-N,N-dimethyl-2-methoxymethylammonium butanoate,

N-ethyl-N,N-dimethyl-2-ethoxymethylammonium butanoate,

N-ethyl-N,N-dimethyl-2-methoxyethylammonium pentanoate,

N-ethyl-N,N-dimethyl-2-ethoxyethylammonium pentanoate,

N-ethyl-N,N-dimethyl-2-methoxymethylammonium pentanoate,

N-ethyl-N,N-dimethyl-2-ethoxymethylammonium pentanoate,

N,N-diethyl-N-methyl-2-methoxyethylammonium propionate,

N,N-diethyl-N-methyl-2-ethoxyethylammonium propionate,

N,N-diethyl-N-methyl-2-methoxymethylammonium propionate,

N,N-diethyl-N-methyl-2-ethoxymethylammonium propionate,

N,N-diethyl-N-methyl-2-methoxyethylammonium butanoate,

N,N-diethyl-N-methyl-2-ethoxyethylammonium butanoate,

N,N-diethyl-N-methyl-2-methoxymethylammonium butanoate,

N,N-diethyl-N-methyl-2-ethoxymethylammonium butanoate,

N,N-diethyl-N-methyl-2-methoxyethylammonium pentanoate,

N,N-diethyl-N-methyl-2-ethoxyethylammonium pentanoate,

N,N-diethyl-N-methyl-2-methoxymethylammonium pentanoate,

N,N-diethyl-N-methyl-2-ethoxymethylammonium pentanoate,

N,N,N-trimethyl-2-methoxyethylammonium propionate,

N,N,N-trimethyl-2-ethoxyethylammonium propionate,

N,N,N-trimethyl-2-methoxymethylammonium propionate,

N,N,N-trimethyl-2-ethoxymethylammonium propionate,

N,N,N-trimethyl-2-methoxyethylammonium butanoate,

N,N,N-trimethyl-2-ethoxyethylammonium butanoate,

N,N,N-trimethyl-2-methoxymethylammonium butanoate,

N,N,N-trimethyl-2-ethoxymethylammonium butanoate,

N,N,N-trimethyl-2-methoxyethylammonium pentanoate,

N,N,N-trimethyl-2-ethoxyethylammonium pentanoate,

N,N,N-trimethyl-2-methoxymethylammonium pentanoate,

N,N,N-trimethyl-2-ethoxymethylammonium pentanoate,

N,N,N-triethyl-2-methoxyethylammonium propionate,

N,N,N-triethyl-2-ethoxyethylammonium propionate,

N,N,N-triethyl-2-methoxymethylammonium propionate,

N,N,N-triethyl-2-ethoxymethylammonium propionate,

N,N,N-triethyl-2-methoxyethylammonium butanoate,

N,N,N-triethyl-2-ethoxyethylammonium butanoate,

N,N,N-triethyl-2-methoxymethylammonium butanoate,

N,N,N-triethyl-2-ethoxymethylammonium butanoate,

N,N,N-triethyl-2-methoxyethylammonium pentanoate,

N,N,N-triethyl-2-ethoxyethylammonium pentanoate,

N,N,N-triethyl-2-methoxymethylammonium pentanoate,

N,N,N-triethyl-2-methoxyethylammonium pentanoate.

A particularly preferred compound is N-ethyl-N,N-dimethyl-2-methoxyethylammonium propionate.

The invention likewise relates to a process for the production of compounds of formula I, as described above, in which ammonium halides of formula II

[NRR¹R²R³]⁺[Hal]⁻ II,

in which [Hal]⁻ is Cl⁻, Br⁻ or I⁻ and R, R¹ to R³ has a meaning as described herein, are converted to hydroxides via ion exchange followed by reaction with the acid R⁴—COOH, in which R⁴ is defined as described herein.

### DETAILED DESCRIPTION OF THE INVENTION

The compounds of formula II are commercially available or may be synthesized by methods which are described, for example, in the standard works, such as Houben-Weyl, Methoden der organischen Chemie (Methods of Organic Chemistry), Georg-Thieme-Verlag, Stuttgart, to be precise under reaction conditions which are known and suitable for the said reactions. Use can also be made here of variants known per se which are not mentioned here in greater detail.

As an example, the ammonium halides of formula H may be synthesized by reaction of corresponding amines NRR¹R² with haloalkanes HalR³ in solvents at temperatures between 10 and 100° C., in which Hal is Cl, Br or I and R, R¹ to R³ has a meaning as described above. Typical solvents are acetonitrile, isopropanol, toluene, heptane or cyclohexane.

A typical ion exchange resin which can be used in the inventive process is Merck Product Nr. 104767 Ion Exchanger III (strongly basic anion exchanger, OH-Form) for Analysis. All other ion exchange resins which have the same properties as this strongly basic anion exchanger can be used.

The ion exchange typically takes place in water as solvent for the ammonium halide. Other useful solvents than water are methanol, ethanol, propanol, butanol, isopropanol and isobutanol. The corresponding solution of the ammonium hydroxide is reacted with R⁴COOH to form the compound of formula I. The substituents R, R¹ to R⁴ have the meaning as described above.

However it is possible to synthesize the corresponding hydroxide via bipolar membrane electrodialysis or via metathesis reaction of ammonium halides with a metal hydroxide such as silver hydroxide, lead hydroxide, potassium hydroxide or sodium hydroxide and to react it with the corresponding acid within the following.

The ionic liquids of formula I may be used in addition as solvent or solvent additive, as phase-transfer catalyst, as extractant, as heat-transfer medium, as surface-active substance, as plasticiser, as flame retardant, as supporting electrolyte in an electrochemical cell or as additive in electrochemical cells.

In the case of the use of the said ionic liquids as solvents, these are suitable in any type of reaction known to the person skilled in the art, for example for transition-metal- or enzyme-catalysed reactions, such as, for example, hydroformylation reactions, oligomerisation reactions, esterifications or isomerisations, where the said list is not exhaustive.

When used as extractant, the ionic liquid can be employed to separate off reaction products, but also to separate off impurities, depending on the solubility of the respective component in the ionic liquid. In addition, the ionic liquids may also serve as separation media in the separation of a plurality of components, for example in the distillative separation of a plurality of components of a mixture.

Further possible applications are use as plasticizer in polymer materials, as flame retardant for a number of materials or applications, and as a supporting electrolyte or additive in various electrochemical cells and applications, for example in galvanic cells, in capacitors or in fuel cells.

Further fields of applications of the new chemical compounds, e.g. ionic liquids, according to this invention are solvents for carbohydrate containing solids in particular biopolymers and derivatives or degredation products thereof. In addition, these new compounds can be applied as lubricants, working fluids for maschines, such as compressors, pumps or hydraulic devices. The ionic liquids according to this invention may be also used in electro-chemical cells in particular for electro-optical cells or as functional materials in sensors.

The most preferred use of ionic liquids of formula I is the use as anti-dandruff compounds as described above.

The ionic liquids according to the present invention can be used as anti-dandruff agents because of their ability to inhibit the growth and/or progeny of Malassezia, preferably Malassezia furfur. Directly or indirectly described substances might in addition improve skin and hair conditions, reduce hair loss, show anti-aging properties or act as antioxidants against the harmful effects caused by oxidants such as reactive oxygen species and light (visible, UV, IR). Exemplary hair conditions include improvements in hair gloss, hair volume and elasticity, in the stability of natural and artificial hair colors and in the inhibition or camouflage of hair-greying. Exemplary skin conditions include the improvements in the skin barrier function, skin hydration, skin oxidation status as well as in the support of a wound healing process.

The ionic liquids according to the present invention show a good microbicidal activity against Malassezia furfur, that means the number of germs in a medium can be reproducibly decreased. In particular the number of microorganisms can be decreased to almost zero within 24 h (starting with an inokulum of ca. 10⁵ microorganisms/ml and a verum test concentration of 1%).

The antifungicidal activity of the compounds according to the present invention can be shown by tests known for a person skilled in the art, for example those based on DIN 58940 and 58944.

The invention relates likewise to a composition comprising at least one compound of formula I as described above.

Therefore, in a preferred embodiment of the invention compositions according to the present invention can be used in the form of solutions, suspensions, emulsions, pastes, ointments, gels, creams, lotions, shampoos, powders, oils, waxes, pencils, shampoos, deodorants-creams, deodorant sticks, roll-ons, sprays, pump sprays, aerosols, polishes, varnishes or hair lacquers.

The compositions of the invention may be preferably in the form of cosmetic, dermatological or pharmaceutical formulations or medicinal products.

Within the following medicinal product shall mean a medical device as defined below.

Directive 2007/47/ec of the European Parliament and of the council of 5 Sep., 2007, which amended the Council Directive 93/42/EEC of 14 Jun., 1993 concerning medical devices, defines a medical device as any instrument, apparatus, appliance, software, material or other article, whether used alone or in combination, including the software intended by its manufacturer to be used specifically for diagnostic and/or therapeutic purposes and necessary for its proper application, intended by the manufacturer to be used for human beings. Devices are to be used for the purpose of:
Diagnosis, prevention, monitoring, treatment or alleviation of disease.
Diagnosis, monitoring, treatment, alleviation of or compensation for an injury or handicap.
Investigation, replacement or modification of the anatomy or of a physiological process
Control of conception

This includes devices that do not achieve its principal intended action in or on the human body by pharmacological, immunological or metabolic means, but which may be assisted in its function by such means.

The compositions according to the invention may include or comprise, essentially consist of or consist of the said necessary or optional constituents. All compounds or components which can be used in the agents or compositions are either known and commercially available or can be synthesised by known processes.

The invention likewise relates to a process for the preparation of a composition as described above, characterised in that at least one compound of formula I as described above is mixed with a carrier and optionally with further active compounds or auxiliaries.

The carriers and optionally the further active compounds or auxiliaries depends on the application field of the inventive composition and are known to the skilled artisan in said field of application.

Preferably the composition comprising at least one compound of formula I according to the present invention is a cosmetic formulation. Cosmetic formulations can be in the form of solutions, suspensions, emulsions, pastes, ointments, gels, creams, lotions, shampoos, powders, oils, waxes, pencils, deodorant-creams, gels, shampoos, lotions, emulsions, deodorant sticks, roll-ons, aerosols, sprays and pump sprays or lacquers, especially nail lacquers.

Depending on the formulation or application the content preferably lies in the range of 0.01% to 10% per weight, preferably in the range of 0.05% to 5% per weight, particularly preferably in the range of 0.1% to 2% per weight, based on the composition in total.

A suitable formulation is in the form of a shampoo or lotion for rinsing out, the formulation in question being applied before or after shampooing, before or after colouring or bleaching or before or after permanent waving. It is also possible to choose a formulation in the form of a lotion or gel for styling or treating the hair, in the form of a lotion or gel for brushing or blow-waving, in the form of a hair lacquer, permanent waving composition, colorant or bleach for the hair. The cosmetic formulation may comprise various adjuvants used in this type of composition, such as surface-active agents, thickeners, polymers, softeners, preservatives, foam stabilizers, electrolytes, organic solvents, silicone derivatives, antigrease agents, dyes and/or pigments which colour the composition itself or the hair, or other ingredients customarily used for hair care.

In all above-mentioned applications the compositions according to the present invention can advantageously be combined with all known anti-dandruff agents, such as climbazole, zinc pyrithione, copper pyrithione or sodium pyrithione in order to boost their individual anti-dandruff activities.

Compositions comprising at least one compound of formula I according to the present invention usually comprise several ingredients. In the following examples of commonly used ingredients, especially for cosmetic formulations, are given.

Preferred formulations or applications additionally comprise at least one insect repellent, preferably selected from the group consisting of ethyl 3-(N-n-butyl-N-acetylamino)propionate, 2-(2-hydroxyethyl)-1-methylpropyl 1-piperidinecarboxylate, N,N-diethyl-3-methylbenzamide, dimethyl phthalate, butopyronoxyl, 2,3,4,5-bis(2-butylene)tetrahydro-2-furaldehyde, N,N-diethylcaprylamide, N,N-diethylbenzamide, o-chloro-N,N-diethylbenzamide, dimethyl carbate, di-n-propyl isocinchomeronate, 2-ethylhexane-1,3-diol, N-octylbicycloheptenedicarboximide or piperonyl butoxide, particularly preferably selected from the group 3-(N-n-butyl-N-acetylamino)propionate, 2-(2-hydroxyethyl)-1-methylpropyl 1-piperidinecarboxylate and N,N-diethyl-3-methylbenzamide.

These insect repellents are generally incorporated into cosmetic formulations in an amount of from 0.5% to 20% by weight, preferably 1%-8% by weight, based on the composition in total.

Preferred formulations or applications additionally comprise at least one UV filter resulting in antimicrobial preparations having light protection properties. The UV filter can preferably be selected from the group of dibenzoylmethane derivatives. The dibenzoylmethane derivatives used within the scope of the present invention are products which are already well known per se and are described, in particular, in the specifications FR-A-2 326 405, FR-A-2 440 933 and EP-A-0 114 607.

In principle, all known UV filters are suitable for combination with dibenzoylmethane derivatives and with the compounds of the formula I according to the invention, for example one or more additional hydrophilic or lipophilic sun-protection filters which are effective in the UV-A region and/or UV-B region and/or IR and/or VIS region (absorbers). These additional filters can be selected, in particular, from cinnamic acid derivatives, salicylic acid derivatives, camphor derivatives, triazine derivatives, β,β-diphenyl acrylate derivatives, p-aminobenzoic acid derivatives and polymeric filters and silicone filters, which are described in the application WO 93/04665. Further examples of organic filters are indicated in Patent Application EP-A 0 487 404. Particular preference is given to UV filters whose physiological acceptability has already been demonstrated. Both for UVA and UVB filters, there are many proven substances which are known from the specialist literature.

These organic UV filters are generally incorporated into cosmetic formulations in an amount of from 0.5% to 10% by weight, preferably 1%-8% by weight, based on the composition in total.

It may furthermore be preferred in accordance with the invention for the preparations to comprise further inorganic UV filters. Preference is given here both to those from the group consisting of titanium dioxides, such as, for example, coated titanium dioxide (for example Eusolex® T-2000 or Eusolex®T-AQUA), zinc oxides (for example Sachtotec®), iron oxides and also cerium oxides. These inorganic UV filters are generally incorporated into cosmetic preparations in an amount of from 0.5% to 20% by weight, preferably 2% -10% by weight, based on the composition in total. In particular, it may be preferred here for a UV-Filter to be incorporated into one phase of an emulsion and a further inorganic UV filter to be incorporated into the other phase.

Furthermore it is preferred to combine compounds of formula I according to the present invention with antioxidants, humectants, vitamins such as panthenol, or vitamin B6 derivatives (e.g. niacin amide) or any derivative of ascorbic acid, skin and hair care actives, in particular watersoluble actives such as carnitine, creatinine, creatine, ectoin, dihydroxyacetone, quaternized actives, and bioflavanoids such as troxerutin or isoquercetin or UV filters like phenyl benzimidazole sulfonic acid.

Preferred auxiliaries are selected from the group consisting of stabilizers, solubilizers, colorants and odor improvers.

Ointments, pastes, creams and gels may comprise the customary excipients, for example animal and vegetable fats, waxes, paraffins, starch, tragacanth, cellulose derivatives, polyethylene glycols, silicones, bentonites, silica, talc and zinc oxide, or mixtures of these substances.

Powders and sprays may comprise the customary excipients, for example lactose, talc, silica, aluminium hydroxide, calcium silicate and polyamide powder, or mixtures of these substances. Sprays may additionally comprise the customary propellants, for example chlorofluorocarbons, propane/butane or dimethyl ether.

Solutions and emulsions may comprise the customary excipients, such as solvents, solubilisers and emulsifiers, for example water, ethanol, isopropanol, ethyl carbonate, ethyl acetate, benzyl alcohol, benzyl benzoate, propylene glycol, 1,3-butyl glycol, oils, in particular cottonseed oil, peanut oil, wheatgerm oil, olive oil, castor oil and sesame oil, glycerol fatty acid esters, polyethylene glycols and fatty acid esters of sorbitan, or mixtures of these substances.

Suspensions may comprise the customary excipients, such as liquid diluents, for example water, ethanol or propylene glycol, suspending agents, for example ethoxylated isostearyl alcohols, polyoxyethylene sorbitol esters and polyoxyethylene sorbitan esters, microcrystalline cellulose, aluminium metahydroxide, bentonite, agar-agar and tragacanth, or mixtures of these substances.

Soaps may comprise the customary excipients, such as alkali metal salts of fatty acids, salts of fatty acid monoesters, fatty acid protein hydrolysates, isethionates, lanolin, fatty alcohol, vegetable oils, plant extracts, glycerol, sugars, or mixtures of these substances.

Surfactant-containing products can comprise the conventional carriers, such as salts of fatty alcohol sulfates, fatty alcohol ether sulfates, sulfosuccinic acid monoesters, fatty acid albumen hydrolysates, isothionates, imidazolinium derivatives, methyl taurates, sarcosinates, fatty acid amide ether sulfates, alkylamidobetaines, fatty alcohols, fatty acid glycerides, fatty acid diethanolamides, vegetable and synthetic oils, lanolin derivatives, ethoxylated glycerol fatty acid esters or mixtures of these substances.

Face and body oils may comprise the customary excipients, such as synthetic oils, such as fatty acid esters, fatty alcohols, silicone oils, natural oils, such as vegetable oils and oily plant extracts, paraffin oils or lanolin oils, or mixtures of these substances.

Further typical cosmetic application forms are also shampoos, lipsticks, lipcare sticks, mascara, eyeliner, eye-shadow, rouge, powder make-up, emulsion make-up and wax make-up, and sunscreen, pre-sun and after-sun preparations.

The preferred preparation forms according to the invention include, in particular, emulsions.

The aqueous phase of the preparations according to the invention optionally advantageously comprises alcohols, diols or polyols having a low carbon number, and ethers thereof, preferably ethanol, isopropanol, propylene glycol, glycerol, ethylene glycol, ethylene glycol monoethyl or monobutyl ether, propylene glycol monomethyl, monoethyl or monobutyl ether, diethylene glycol monomethyl or monoethyl ether and analogous products, furthermore alcohols having a low carbon number, for example ethanol, isopropanol, 1,2-propanediol or glycerol, and, in particular, one or more thickeners, which may advantageously be selected from the group consisting of silicon dioxide, aluminium silicates, polysaccharides and derivatives thereof, for example hyaluronic acid, xanthan gum, hydroxypropylmethylcellulose, particularly advantageously from the group consisting of the polyacrylates, preferably a polyacrylate from the group consisting of the so-called Carbopols, for example Carbopol grades 980, 981, 1382, 2984 or 5984, in each case individually or in combination.

Cosmetic and dermatological preparations according to the invention may exist in various forms. Thus, they may be, for example, a solution, a water-free preparation, an emulsion or microemulsion of the water-in-oil (W/O) or oil-in-water (O/W) type, a multiple emulsion, for example of the water-in-oil-in-water (W/O/W) type, a gel, a solid stick, an ointment or an aerosol. It is also advantageous to administer ectoin or hydroxyectoin in encapsulated form, for example in collagen matrices and other conventional encapsulation materials, for example as cellulose encapsulations, in gelatine, wax matrices or liposomally encapsulated. In particular, wax matrices, as described in DE-A 43 08 282, have proven favourable. Preference is given to emulsions. O/W emulsions are particularly preferred. Emulsions, W/O emulsions and O/W emulsions are obtainable in a conventional manner.

Emulsifiers that can be used are, for example, the known W/O and O/W emulsifiers. It is advantageous to use further conventional co-emulsifiers in the preferred O/W emulsions according to the invention. The commercially available product Ceralution C (Sasol) has to be proven to be in particular advantageous as emulsifier.

Co-emulsifiers which are advantageous according to the invention are, for example, O/W emulsifiers, principally from the group consisting of the substances having HLB values of 11-16, very particularly advantageously having HLB values of 14.5-15.5, so long as the O/W emulsifiers have saturated radicals R and R′. If the O/W emulsifiers have unsaturated radicals R and/or R′ or in the case of isoalkyl derivatives, the preferred HLB value of such emulsifiers may also be lower or higher.

The preparation may comprise cosmetic adjuvants which are usually used in this type of preparation, such as, for example, thickeners, softeners, moisturisers, surface-active agents, emulsifiers, preservatives, antifoams, perfumes, waxes, lanolin, propellants, dyes and/or pigments which colour the composition itself or the skin, and other ingredients usually used in cosmetics.

Emulsions according to the invention are advantageous and comprise, for example, the said fats, oils, waxes and other fatty substances, as well as water and an emulsifier, as usually used for a preparation of this type.

The lipid phase may advantageously be selected from the following group of substances:
mineral oils, mineral waxes;
oils, such as triglycerides of capric or caprylic acid, furthermore natural oils, such as, for example, castor oil;
fats, waxes and other natural and synthetic fatty substances, preferably esters of fatty acids with alcohols having a low carbon number, for example with isopropanol, propylene glycol or glycerol, or esters of fatty alcohols with alkanoic acids having a low carbon number or with fatty acids;
silicone oils, such as dimethylpolysiloxanes, diethylpolysiloxanes, diphenylpolysiloxanes and mixed forms thereof.

For the purposes of the present invention, the oil phase of the emulsions, oleogels or hydrodispersions or lipodispersions is advantageously selected from the group consisting of esters of saturated and/or unsaturated, branched and/or unbranched alkanecarboxylic acids having a chain length of from 3 to 30 carbon atoms and saturated and/or unsaturated, branched and/or unbranched alcohols having a chain length of from 3 to 30 carbon atoms, or from the group consisting of esters of aromatic carboxylic acids and saturated and/or unsaturated, branched and/or unbranched alcohols having a chain length of from 3 to 30 carbon atoms. Ester oils of this type can then advantageously be selected from the group consisting of isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, oleyl oleate, oleyl erucate, erucyl oleate, erucyl erucate and synthetic, semi-synthetic and natural mixtures of esters of this type, for example jojoba oil.

The oil phase may furthermore advantageously be selected from the group consisting of branched and unbranched hydrocarbons and waxes, silicone oils, dialkyl ethers, or the group consisting of saturated and unsaturated, branched and unbranched alcohols, and fatty acid triglycerides, specifically the triglycerol esters of saturated and/or unsaturated, branched and/or unbranched alkanecarboxylic acids having a chain length of from 8 to 24, in particular 12-18, carbon atoms. The fatty acid triglycerides may advantageously be selected, for example, from the group consisting of synthetic, semi-synthetic and natural oils, for example olive oil, sunflower oil, soya oil, peanut oil, rapeseed oil, almond oil, palm oil, coconut oil, palm kernel oil and the like.

Any desired mixtures of oil and wax components of this type may also advantageously be employed for the purposes of the present invention. It may also be advantageous to employ waxes, for example cetyl palmitate, as the only lipid component of the oil phase.

The entire disclosure of all applications, patents and publications, cited above are hereby incorporated by reference.

The compounds and their production process as well as compositions according to the present invention is more illustratively demonstrated but not limited by means of the following examples.

### EXAMPLES

### Example 1

### Synthesis of N-ethyl-N,N-dimethyl-2-methoxyethylammonium Propionate (1136)

A. Alkylation

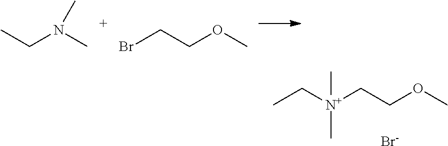

A solution of 200 ml N,N-dimethylethylamine in 250 mL acetonitrile is mixed with mechanical stirring and heated to 80° C. To this solution, 282 g 2-bromoethylmethyl ether is added slowly while stirring and the reaction mixture is stirred for 3 h at 80° C.

After 3 h, the reaction mixture is added to 2,5 L ethylacetate, which resulted in the precipitation of the bromide product. After filtration and drying, 343 g of a white solid is obtained.

B. Anion Exchange

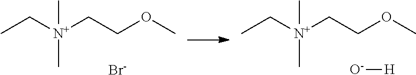

0.77 kg Merck Product Nr. 104767 Ion Exchanger III (strongly basic anion exchanger, OH-Form) for Analysis is washed with water in a type G3 glass filter and dried. Afterwards, a solution of 130 g N-ethyl-N,N-dimethyl-2-methoxyethylammonium bromide in 500 ml water is added. The resulting aqueous solution of N-ethyl-N,N-dimethyl-2-methoxyethylammonium hydroxide is separated from the ion exchange resin via filtration. The pH of the solution after ion exchange is about pH 14, and the bromide content is about 181 mg/L

C. Neutralization with Propionic Acid

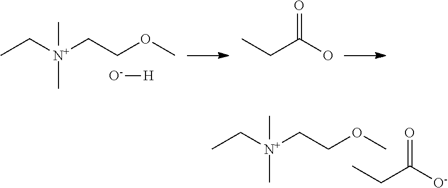

While stirring, 12.5 mL propionic acid is added to 256 ml aqueous solution of N-ethyl-N,N-dimethyl-2-methoxyethylammonium hydroxide at room temperature. After distillation of the solvent, 35 g of N-ethyl-N,N-dimethyl-2-methoxyethylammonium propionate is obtained as clear liquid. 1H NMR (d6-DMSO): δ=3.74 (m, 2H), 3.54 (t, 2H), 3.42 (q, 2H), 3.30 (s, 3H), 3.05 (s, 6H), 1.78 (q, 3H), 1.24 (t, 3H), 0.86 (t, 3H).

### Example 2

### Antimicrobial Efficacy was Tested using the Following Challenge Testing Procedure

The challenge testing procedure consists in challenging a non-contaminated antimicrobial product according to the invention with a prescribed inoculum of suitable microorganisms and storing the inoculated product at a prescribed temperature, e.g. room temperature. The number of organisms surviving in the test products is determined at specified intervals of time.

According to the description given in the European pharmacopea (Ph.Eur.5 Ausgabe, Grundwerk 2005, §5.1.3), the challenge tests are done following the procedure given thereafter.

Production of the Inoculum

A fresh stock culture of the specific micro-organsisms is inoculated on the surface of Agar medium B for bacteria and on the surface of Agar medium C for fungi. The bacteria culture will be incubated until sufficient sporulation (18-24h at 30-35° C.) In order to harvest the bacteria, the surface of the Agar media is washed out with a sterile solution containing sodium chloride (9 g/l) and poured into an adequate vessel. The concentration of germs in the suspension will be adjusted with the same solution to a concentration closed to 10⁸ micro-organism/ml. Immediately after that action, a sample of this suspension is taken and the germ concentration in CFU/ml will be measured thanks to the method of membran filtration or counting on Agar plate. This value serves determining the value of the inoculum. The suspension must be used immediately.

Method of Determination of Germ Count

In order to determine the number of micro-organisms in the inoculated preparation (containing also the antimicrobial product according to invention), the same Agar medium as for the preparation of the inoculum will be used.

The aqueous suspension/solution containing 1% of the used ionic liquids (=verum) is inoculated with a suspension of the tests micro-organisms (Malassezia furfur) in such a way that a concentration from 10⁵ to 10⁶ microorganism/ml of the preparation is reached. The inoculated volume should not be above 1% v/v of the overall test solution. The suspension will be mixed in order to get a good homogenisation.

Water alone (=placebo) is treated in the same way. Antifungicidial activity of the verum will be detected once the survival rate of a microorganism for the verum is significantely lower than for the placebo.

The inoculated preparation is stored away from the daylight at 20-25° C. In order to begin the test, 1 g or 1 ml samples from the tests preparation (containing the inoculated micro-organsisms and the antimicrobial product according to the invention) will be taken at different intervals of time (in our case , 0.2 min, 5 min, 30 min, 1 h, 3 h, 6 h, 24 h) and the number of microorganisms will be measured using the Agar plate or the membrane filtration method. It is important to make sure that any remaining antimicrobial activity to be eliminated by dilution, filtration or specific inactivation.

Results:

N-ethyl-N,N-dimethyl-2-methoxyethylammonium propionate=1136

The compound shows an unique antifungicidial activity against Malassezia furfur as documented in the following tables (the 1^{st} part of each individual table represents the total number of microorganisms versus incubation time at linear scale, the 2^{nd} part at log-scale):

Malassezia furfur

| | Placebo Water | 1136 [1.0%] |
|---|---|---|
| 0 h | 56000 | 45000 |
| 1 h | 46000 | 41000 |
| 3 h | 68000 | 36000 |
| 6 h | 43000 | 33000 |
| 24 h | 36000 | 100 |
| 0 h | 4.7 | 4.7 |
| 1 h | 4.7 | 4.6 |
| 3 h | 4.8 | 4.6 |
| 6 h | 4.6 | 4.5 |
| 24 h | 4.6 | 2.0 |

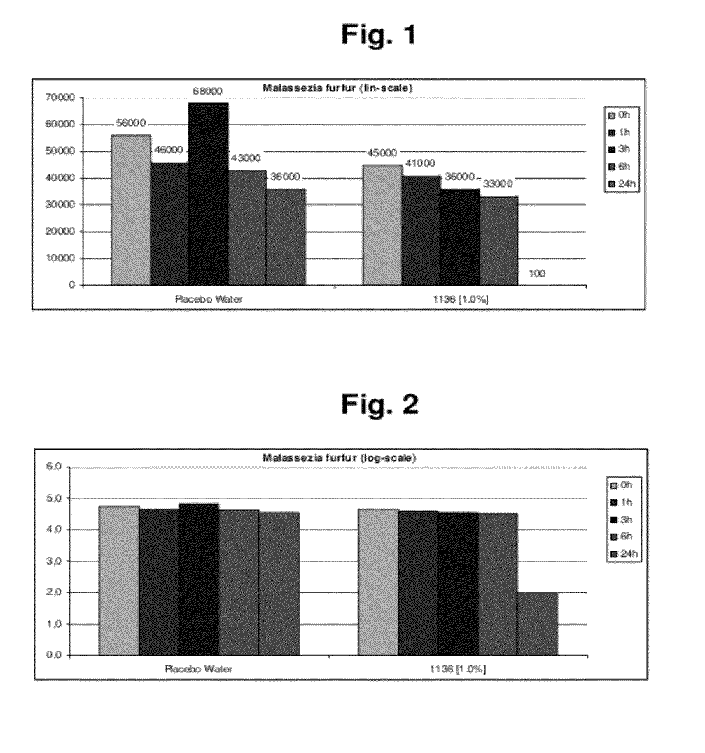

FIG. 1 depicts the linear scale. FIG. 2 depicts the log scale.

Since activity against Malassezia furfur plays a key role for the performance of anti-dandruff compositions, 1136 is in particular suitable to be incorporated into such product formulations. In addition to such product formulations it may be of particular interest that 1136 selectively inhibit the growth of the undesired microorganism Malassezia furfur of the natural microflora of the skin whereas the growth of desired microorganisms may be not or minor affected (e.g. Staphylococcus epiderimidis).

The following table shows the results for 1136 against Staphylococcus epidermidis in the above mentioned test system:

Staphylococcus epidermidis

| | Placebo Water | 1136 [1.0%] |
|---|---|---|
| 0 h | 160000 | 210000 |
| 1 h | 150000 | 160000 |
| 3 h | 190000 | 180000 |
| 6 h | 120000 | 170000 |
| 24 h | 770000 | 22000 |
| 0 h | 5.2 | 5.3 |
| 1 h | 5.2 | 5.2 |
| 3 h | 5.3 | 5.3 |
| 6 h | 5.1 | 5.2 |
| 24 h | 5.9 | 4.3 |

Application (Formulation) Examples:

A) Pump hairspray (amounts are given in % by weight of the formulation):

| | 1 | 2 | 3 |
|---|---|---|---|
| A | | | |
| 1136 | 0.1 | 0.3 | 0.5 |
| Ethanol 96% pure | Ad 100 | Ad 100 | Ad 100 |
| PVP/VA copolymer | 6 | 6 | 6 |
| PVP/VA W 735 | | | |
| B | | | |
| Diethylhexyl Syringylidenemalonate, | 0.06 | 0.25 | 0.5 |
| Caprylic/Capric Triglyceride | | | |
| (Oxynex ® ST Liquid) | | | |
| PEG-75 Lanolin | 0.2 | 0.2 | 0.2 |
| BHT | | | |
| (Solan E-Low Dioxane) | | | |
| Parfum | 0.1 | 0.1 | 0.1 |
| (Frag 280853 Green Activating) | | | |
| C | | | |
| Aqua (Water) | 13 | 13 | 13 |
| Titriplex III (sodium EDTA) | 0.1 | 0.1 | 0.1 |
| PEG-12 dimethicone | 0.5 | 0.5 | 0.5 |
| Dow Corning 193 Fluid | | | |
| 0.1% D&C Red No 33 (CI 17200) in | 0.2 | 0.2 | 0.2 |
| water | | | |
| PEG-40 Hydrogenated Castor Oil | 1 | 1 | 1 |
| (Cremophor RH 410) | | | |

Procedure: Phase B is added to phase A while stirring. Phase C is mixed and added to the combined phases A and B while stirring until homogeneity.

The above formulation may of course contain one or more actives among the following list: Zinc Pyrithione, Piroctone Olamine, Ketoconazole, Tropolone, Hinokitol Selenium Sulfide, Salicylic Acid, Climbazole, Sodium Salicylate, Ciclopiroxolamine, Neem, Basilic oil, Ichtammol, Melaleuca Alternifolia, Centaurea Cyanus, Melia Azadirachta, Farnesol, Sulfur, Clotrimazole, Crotamiton, Zinc Salicylate, Tussilago farfara, Arctium lappa, Zinc Sulfate, Rosmarinus officinalis, Ketoconazole.

B) W/O emulsions (amounts are given in % by weight of the formulation)

| Emulsion | A | B | C | D | E | F |
|---|---|---|---|---|---|---|
| Polyglyceryl-2- | 3 | 5 | 3 | | | |
| Dipolyhydroxystearate | | | | | | |
| PEG-30 Dipolyhydroxystearate | | | 2 | 3 | 4 | 5 |
| Sodium starch Octenylsuccinate | 0.5 | 0.4 | | 0.3 | | 1 |
| Glycine | 0.3 | 0.3 | 0.5 | 0.4 | | |
| Alcohol | | 5 | 2 | 5 | 4 | |
| Magnesium sulfate | 0.2 | 0.3 | 0.3 | 0.4 | 0.5 | 0.2 |
| C₁₂₋₁₅ Alkyl Benzoate | 5 | 3 | | | 5 | |
| C₁₂₋₁₃ Alkyl Tartrate | | 2 | | | | |
| Butylene glycol | 5 | | | | 3 | 3 |
| Dicaprylate/Dicaprate | | | | | | |
| Dicaprylyl Ether | | | | | 2 | |
| Mineral oil | | 4 | | 6 | | 8 |
| Octyldodecanol | 2 | | | | | |
| Dicaprylcaprate | | 2 | | | 2 | 2 |
| Cyclomethicone | 5 | | 5 | 10 | | |
| Dimethicone | | | | 5 | | |
| Isohexadecane | | 1 | | | | |
| Butylene glycol | 5 | 8 | | | | 3 |
| Propylene glycol | | | 1 | | 5 | 3 |
| Glycerol | 3 | 5 | 7 | 10 | 3 | 3 |
| C18-38 Acid triglyceride | 0.5 | | 1 | | 1 | |
| Titanium dioxide | 5 | 6 | 4 | | | 4 |
| Zinc oxide | 5 | | | | | |
| Bis-Ethylhexyloxyphenol | | 3 | 3 | 2 | | |
| Methoxyphenyltriazin | | | | | | |
| Ethylhexyl triazone | | 4.5 | 3 | | 3 | |
| 1136 | 0.2 | 0.5 | 0.5 | 0.5 | 1 | 1.5 |
| Diethylhexyl butamidotriazone | | | 1.5 | 4 | | |
| Butyl | 2 | 3 | 4 | | 1 | 3 |
| Methoxydibenzoylmethane | | | | | | |
| Uvinul ® A Plus | | | | 4 | 2 | |
| Ethylhexyl methoxycinnamate | | | | | 7 | 5 |
| Benzotriazole coppled to | 4 | | 6 | | | |
| gelatine | | | | | | |
| Taurine | 0.1 | | | 0.5 | 0.2 | |
| Vitamin E Acetate | 0.2 | 02 | | 0.3 | 0.1 | 0.5 |
| Na₂H₂EDTA | 0.1 | 0.1 | 0.2 | 0.2 | 0.2 | 0.5 |
| C8-C16 Alkylpolyglycoside | 1 | | | | | |
| Parfum, preservative | q.s. | q.s | q.s | q.s | qs. | qs. |
| Dye. | q.s. | q.s. | q.s. | q.s | q.s. | q.s. |
| Sodium hydroxid | q.s. | q.s. | q.s. | q.s | q.s. | q.s. |
| Water | ad 100 | ad 100 | ad 100 | ad 100 | ad 100 | ad 100 |

The above formulation may of course contain one or more actives among the following list: Zinc Pyrithione, Piroctone Olamine, Ketoconazole, Tropolone, Hinokitol, Selenium Sulfide, Salicylic Acid, Climbazole, Sodium Salicylate, Ciclopiroxolamine, Neem, Basilic oil, Ichtammol, Melaleuca Alternifolia, Centaurea Cyanus, Melia Azadirachta, Farnesol, Sulfur , Clotrimazole, Crotamiton, Zinc Salicylate, Tussilago farfara, Arctium lappa, Zinc Sulfate, Rosmarinus officinalis, Myrtrimonium Bromid, Lactic Acid, Chlorohexidine Digluconate, Phenoxyisopropanol, Isopropanol, Farnesol, Glycolic Acid, Tannic acid, Alcohol, Triclosan, Zinc Gluconate, Zinc PCA, Camphor, Aluminium salts, Sodium Lactate, Polyaminopropyl Biguanide, Zinc Acetat, Triethyl Citrate, Ethylhexylglycerol, Aluminium Circonium, Tetrachlorohydrex GLY, Pentetic Acid, Diisopropylamine Aminoethylpropanol, Zinc Ricinoleate, Aluminium Sesquichlorohydrate, Lactic Acid, Triclosan.

C) hair care formulation (amounts are given in % by weight of the formulation)

| Ingredient | A | B | C | D | E | F |
|---|---|---|---|---|---|---|
| Disodium EDTA | 0.1 | 0.1 | 0.1 | 0.1 | 0.1 | 0.1 |
| Oxynex ®ST | 2 | 2 | 2 | 2 | 2 | 2 |
| 1136 | 0.01 | 0.05 | 0.25 | 0.5 | 1 | 2 |
| Hexamidine diisethionate | 0.1 | 0 | 0 | 0 | 0 | 0 |
| Tetrahydrocurcumin | 0 | 0.5 | 0 | 0 | 0 | 0 |
| Glycyrrhetinic acid | 0 | 0 | 0.3 | 0 | 0 | 0 |
| Thiotaine ® | 0 | 0 | 0 | 5 | 0 | 0 |
| N-undecylenoyl-L-phenylalanine | 0 | 0 | 0 | 0 | 1 | 0 |
| N-acetyl glucosamine | 0 | 0 | 0 | 0 | 0 | 2 |
| Niacinamide | 5 | 5 | 5 | 5 | 5 | 5 |
| Citric acid | 0.015 | 0 | 0 | 0 | 0 | 0 |
| Isohexadecane | 3 | 3 | 3 | 3 | 3 | 3 |
| Isopropyl isostearate | 1.33 | 1.33 | 1.33 | 1.33 | 1.33 | 1.33 |
| Isopropyl N-laurosylsarcosinate | 0 | 0 | 5 | 0 | 0 | 0 |
| Sucrose polycottonseedate | 0.67 | 0.67 | 0.67 | 0.67 | 0.67 | 0.67 |
| Polymethylsilsesquioxane | 0.25 | 0.25 | 0.25 | 0.25 | 0.25 | 0.25 |
| Cetearyl glucoside + | 0.2 | 0.2 | 0.2 | 0.2 | 0.2 | 0.2 |
| cetearyl alcohol | | | | | | |
| Behenyl alcohol | 0.4 | 0.4 | 0.4 | 0.4 | 0.4 | 0.4 |
| Ethylparaben | 0.2 | 0.2 | 0.2 | 0.2 | 0.2 | 0.2 |
| Propylparaben | 0.1 | 0.1 | 0.1 | 0.1 | 0.1 | 0.1 |
| Cetyl alcohol | 0.32 | 0.32 | 0.32 | 0.32 | 0.32 | 0.32 |
| Stearyl alcohol | 0.48 | 0.48 | 0.48 | 0.48 | 0.48 | 0.48 |
| Tocopheryl acetate | 0.5 | 0.5 | 0.5 | 0.5 | 0.5 | 0.5 |
| PEG-100 stearate | 0.1 | 0.1 | 0.1 | 0.1 | 0.1 | 0.1 |
| Glycerol | 7 | 7 | 7 | 7 | 7 | 7 |
| Titanium dioxide | 0.6 | 0.6 | 0.6 | 0.6 | 0.6 | 0.6 |
| Polyacrylamide + C13-14 | 3 | 2 | 2 | 2 | 2 | 2 |
| isoparaffin + Iaureth-7 | | | | | | |
| Panthenol | 1 | 1 | 1 | 1 | 1 | 1 |
| Benzyl alcohol | 0.4 | 0.4 | 0.4 | 0.4 | 0.4 | 0.4 |
| Dimethicone + dimethiconol | 2 | 2 | 2 | 2 | 2 | 2 |
| Water | to 100 | to 100 | to 100 | to 100 | to 100 | to 100 |

Further Formulation Examples for Hair Care Applications

D) The formulations in the table below contain 1136 in the following amounts: Formulation A (0.25% 1136), formulation B (0.5% 1136), formulation C (1.0% 1136), formulation D (2.0% 1136), formulation E (3.0% 1136), formulation F (4.0% 1136).

The formulations A to F may of course contain one or more actives among the following list: Zinc Pyrithione, Piroctone Olamine, Ketoconazole, Tropolone, Hinokitol, Selenium Sulfide, Salicylic Acid, Climbazole, Sodium Salicylate, Ciclopiroxolamine, Neem, Basilic oil, Ichtammol, Melaleuca Alternifolia, Centaurea Cyanus, Melia Azadirachta, Farnesol, Sulfur , Clotrimazole, Crotamiton, Zinc Salicylate, Tussilago farfara, Arctium lappa, Zinc Sulfate, Rosmarinus officinalis, Myrtrimonium Bromid, Lactic Acid, Chlorohexidine Digluconate, Phenoxyisopropanol, Isopropanol, Farnesol, Glycolic Acid, Tannic acid, Alcohol, Triclosan, Zinc Gluconate, Zinc PCA, Camphor, Aluminium salts, Sodium Lactate, Polyaminopropyl Biguanide, Zinc Acetat, Triethyl Citrate, Ethylhexylglycerol, Aluminium Circonium, Tetrachlorohydrex GLY, Pentetic Acid, Diisopropylamine Aminoethylpropanol, Zinc Ricinoleate, Aluminium Sesquichlorohydrate, Lactic Acid, Triclosan.

Content in formulation (g component per 100 g formulation)

| Component | A | B | C | D | E | F |
|---|---|---|---|---|---|---|
| Disodium EDTA | 0.100 | 0.100 | 0.100 | 0.100 | 0.100 | 0.100 |
| Oxynex ® | 2.000 | 2.000 | 2.000 | 2.000 | 2.000 | 2.000 |
| Hexamidine diisethionate | 0.100 | 0 | 0 | 0 | 0 | 0 |
| Tetrahydrocurcumin | 0 | 0.500 | 0 | 0 | 0 | 0 |
| Glycyrrhetinic acid | 0 | 0 | 0.300 | 0 | 0 | 0 |
| Thiotaine ®¹ | 0 | 0 | 0 | 5.000 | 0 | 0 |
| N-undecylenoyl-L-phenylalanine | 0 | 0 | 0 | 0 | 1.000 | 0 |
| N-acetyl glucosamine | 0 | 0 | 0 | 0 | 0 | 2.000 |
| Niacinamide | 5.000 | 5.000 | 5.000 | 5.000 | 5.000 | 5.000 |
| Citric acid | 0.015 | 0 | 0 | 0 | 0 | 0 |
| Isohexadecane | 3.000 | 3.000 | 3.000 | 3.000 | 3.000 | 3.000 |
| Isopropyl isostearate | 1.330 | 1.330 | 1.330 | 1.330 | 1.330 | 1.330 |
| Isopropyl N-laurosylsarcosinate | 0 | 0 | 5.000 | 0 | 0 | 0 |
| Sucrose polycottonseedate | 0.670 | 0.670 | 0.670 | 0.670 | 0.670 | 0.670 |
| Polymethylsilsesquioxane | 0.250 | 0.250 | 0.250 | 0.250 | 0.250 | 0.250 |
| Cetearyl glucoside + | 0.200 | 0.200 | 0.200 | 0.200 | 0.200 | 0.200 |
| cetearyl alcohol | | | | | | |
| Behenyl alcohol | 0.400 | 0.400 | 0.400 | 0.400 | 0.400 | 0.400 |
| Ethylparaben | 0.200 | 0.200 | 0.200 | 0.200 | 0.200 | 0.200 |
| Propylparaben | 0.100 | 0.100 | 0.100 | 0.100 | 0.100 | 0.100 |
| Cetyl alcohol | 0.320 | 0.320 | 0.320 | 0.320 | 0.320 | 0.320 |
| Stearyl alcohol | 0.480 | 0.480 | 0.480 | 0.480 | 0.480 | 0.480 |
| Tocopheryl acetate | 0.500 | 0.500 | 0.500 | 0.500 | 0.500 | 0.500 |
| PEG-100 stearate | 0.100 | 0.100 | 0.100 | 0.100 | 0.100 | 0.100 |
| Glycerin | 7.000 | 7.000 | 7.000 | 7.000 | 7.000 | 7.000 |
| Titanium dioxide | 0.604 | 0.604 | 0.604 | 0.604 | 0.604 | 0.604 |
| Polyacrylamide + C13-14 | 3.000 | 2.000 | 2.000 | 2.000 | 2.000 | 2.000 |
| isoparaffin + laureth-7 | | | | | | |
| Panthenol | 1.000 | 1.000 | 1.000 | 1.000 | 1.000 | 1.000 |
| Benzyl alcohol | 0.400 | 0.400 | 0.400 | 0.400 | 0.400 | 0.400 |
| Dimethicone + dimethiconol | 2.000 | 2.000 | 2.000 | 2.000 | 2.000 | 2.000 |
| Water (to 100 g) | to 100 | to 100 | to 100 | to 100 | to 100 | to 100 |
| TOTAL | 100 | 100 | 100 | 100 | 100 | 100 |

E) The below formulations contain 1136 in the following amounts: Formulation G (0.01% 1136), formulation H (0.05% 1136), formulation I (0.1% 1136).

The formulations G to I may of course contain one or more actives among the following list: Zinc Pyrithione, Piroctone Olamine, Ketoconazole, Tropolone, Hinokitol, Selenium Sulfide, Salicylic Acid, Climbazole, Sodium Salicylate, Ciclopiroxolamine, Neem, Basilic oil, Ichtammol, Melaleuca Alternifolia, Centaurea Cyanus, Melia Azadirachta, Farnesol, Sulfur , Clotrimazole, Crotamiton, Zinc Salicylate, Tussilago farfara, Arctium lappa, Zinc Sulfate, Rosmarinus officinalis, Myrtrimonium Bromid, Lactic Acid, Chlorohexidine Digluconate, Phenoxyisopropanol, Isopropanol, Farnesol, Glycolic Acid, Tannic acid, Alcohol, Triclosan, Zinc Gluconate, Zinc PCA, Camphor, Aluminium salts, Sodium Lactate, Polyaminopropyl Biguanide, Zinc Acetat, Triethyl Citrate, Ethylhexylglycerol, Aluminium Circonium, Tetrachlorohydrex GLY, Pentetic Acid, Diisopropylamine Aminoethylpropanol, Zinc Ricinoleate, Aluminium Sesquichlorohydrate, Lactic Acid, Triclosan.

Content in formulation (g component per 100 g formulation)

| Component | G | H | I |
|---|---|---|---|
| Disodium EDTA | 0.100 | 0.100 | 0.100 |
| Oxynex ® | 2.000 | 2.000 | 2.000 |
| Cetyl pyridinium chloride | 0.200 | 0 | 0 |
| Pitera ® | 0 | 10 | 0 |
| Ascorbyl glucoside | 0 | 0 | 2.000 |
| Niacinamide | 5.000 | 5.000 | 5.000 |
| Polyquaternium 37 | 0 | 0 | 0 |
| Isohexadecane | 3.000 | 3.000 | 3.000 |
| Isopropyl isostearate | 1.330 | 1.330 | 1.330 |
| Sucrose polycottonseedate | 0.670 | 0.670 | 0.670 |
| Polymethylsilsesquioxane | 0.250 | 0.250 | 0.250 |
| Cetearyl glucoside + cetearyl alcohol | 0.200 | 0.200 | 0.200 |
| Behenyl alcohol | 0.400 | 0.400 | 0.400 |
| Ethylparaben | 0.200 | 0.200 | 0.200 |
| Propylparaben | 0.100 | 0.100 | 0.100 |
| Cetyl alcohol | 0.320 | 0.320 | 0.320 |
| Stearyl alcohol | 0.480 | 0.480 | 0.480 |
| Tocopheryl acetate | 0.500 | 0.500 | 0.500 |
| PEG-100 stearate | 0.100 | 0.100 | 0.100 |
| Glycerin | 7.000 | 7.000 | 7.000 |
| Titanium dioxide | 0.604 | 0.604 | 0.604 |
| Polyacrylamide + C13-14 isoparaffin + | 2.000 | 2.000 | 2.000 |
| laureth-7 | | | |
| Panthenol | 1.000 | 1.000 | 1.000 |
| Benzyl alcohol | 0.400 | 0.400 | 0.400 |
| Dimethicone + dimethiconol | 2.000 | 2.000 | 2.000 |
| Water (to 100 g) | to 100 | to 100 | to 100 |
| TOTAL | 100 | 100 | 100 |

1. Compound of the formula I comprising:
[NRR¹R²R³]⁺[R⁴—COO]⁻ I
wherein
R is methoxyethyl, methoxymethyl, ethoxyethyl or ethoxymethyl,
R¹ to R³ are independantly of each other methyl or ethyl,
R⁴ is linear or branched alkyl with 2 to 4 C atoms.

2. The compound according to claim 1 wherein R is methoxyethyl or ethoxyethyl.

3. The compound according to claim 1 or 2 wherein R⁴ is ethyl.

4. The compound according to any one of claims 1 to 3, wherein:
R is methoxylmethyl, methoxyethyl, ethoxymethyl or ethoxyethyl; R¹, R² and R³ are independently methyl or ethyl; and R⁴ is ethyl, propyl, or butyl;
R is methoxylmethyl, methoxyethyl, ethoxymethyl or ethoxyethyl; R¹ and R² are methyl; R³ is ethyl; and R⁴ is ethyl, propyl, or butyl;
R is methoxylmethyl, methoxyethyl, ethoxymethyl or ethoxyethyl; R¹ and R² are ethyl;
R³ is methyl; and R⁴ is ethyl, propyl, or butyl;
R is methoxylmethyl, methoxyethyl, ethoxymethyl or ethoxyethyl; R¹, R² and R³ are methyl; and R⁴ is ethyl, propyl, or butyl; or
R is methoxylmethyl, methoxyethyl, ethoxymethyl or ethoxyethyl; R¹, R² and R³ are ethyl; and R⁴ is ethyl, propyl, or butyl.

5. The compound according to any one of claims 1 to 4 comprising
N-ethyl-N,N-dimethyl-2-methoxyethylammonium propionate,
N-ethyl-N,N-dimethyl-2-ethoxyethylammonium propionate,
N-ethyl-N,N-dimethyl-2-methoxymethylammonium propionate,
N-ethyl-N,N-dimethyl-2-ethoxymethylammonium propionate,
N-ethyl-N,N-dimethyl-2-methoxyethylammonium butanoate,
N-ethyl-N,N-dimethyl-2-ethoxyethylammonium butanoate,
N-ethyl-N,N-dimethyl-2-methoxymethylammonium butanoate,
N-ethyl-N,N-dimethyl-2-ethoxymethylammonium butanoate,
N-ethyl-N,N-dimethyl-2-methoxyethylammonium pentanoate,
N-ethyl-N,N-dimethyl-2-ethoxyethylammonium pentanoate,
N-ethyl-N,N-dimethyl-2-methoxymethylammonium pentanoate,
N-ethyl-N,N-dimethyl-2-ethoxymethylammonium pentanoate,
N,N-diethyl-N-methyl-2-methoxyethylammonium propionate,
N,N-diethyl-N-methyl-2-ethoxyethylammonium propionate,
N,N-diethyl-N-methyl-2-methoxymethylammonium propionate,
N,N-diethyl-N-methyl-2-ethoxymethylammonium propionate,
N,N-diethyl-N-methyl-2-methoxyethylammonium butanoate,
N,N-diethyl-N-methyl-2-ethoxyethylammonium butanoate,
N,N-diethyl-N-methyl-2-methoxymethylammonium butanoate,
N,N-diethyl-N-methyl-2-ethoxymethylammonium butanoate,
N,N-diethyl-N-methyl-2-methoxyethylammonium pentanoate,
N,N-diethyl-N-methyl-2-ethoxyethylammonium pentanoate,
N,N-diethyl-N-methyl-2-methoxymethylammonium pentanoate,
N,N-diethyl-N-methyl-2-ethoxymethylammonium pentanoate,
N,N,N-trimethyl-2-methoxyethylammonium propionate,
N,N,N-trimethyl-2-ethoxyethylammonium propionate,
N,N,N-trimethyl-2-methoxymethylammonium propionate,
N,N,N-trimethyl-2-ethoxymethylammonium propionate,
N,N,N-trimethyl-2-methoxyethylammonium butanoate,
N,N,N-trimethyl-2-ethoxyethylammonium butanoate,
N,N,N-trimethyl-2-methoxymethylammonium butanoate,
N,N,N-trimethyl-2-ethoxymethylammonium butanoate,
N,N,N-trimethyl-2-methoxyethylammonium pentanoate,
N,N,N-trimethyl-2-ethoxyethylammonium pentanoate,
N,N,N-trimethyl-2-methoxymethylammonium pentanoate,
N,N,N-trimethyl-2-ethoxymethylammonium pentanoate,
N,N,N-triethyl-2-methoxyethylammonium propionate,
N,N,N-triethyl-2-ethoxyethylammonium propionate,
N,N,N-triethyl-2-methoxymethylammonium propionate,
N,N,N-triethyl-2-ethoxymethylammonium propionate,
N,N,N-triethyl-2-methoxyethylammonium butanoate,
N,N,N-triethyl-2-ethoxyethylammonium butanoate,
N,N,N-triethyl-2-methoxymethylammonium butanoate,
N,N,N-triethyl-2-ethoxymethylammonium butanoate,
N,N,N-triethyl-2-methoxyethylammonium pentanoate,
N,N,N-triethyl-2-ethoxyethylammonium pentanoate,
N,N,N-triethyl-2-methoxymethylammonium pentanoate,
N,N,N-triethyl-2-methoxyethylammonium pentanoate.

6. A composition comprising at least one compound of formula I according to any one of claims 1 to 5.

7. The composition according to claim 6 further comprising another anti-dandruff agent, active compound, insect repellent, organic UV filter, inorganic UV filter, antioxidant, humectant, vitamin, hair care active dibenzoylmethane derivative, stabilizer, solubilizer, colorant, odor improver, or a combination thereof.

8. The composition according to claim 6, wherein
the other anti-dandruff agent is selected from the group consisting of climbazole, zinc pyrithione, copper pyrithione and sodium pyrithione;
the insect repellent is selected from the group consisting of ethyl 3-(N-n-butyl-N-acetylamino)propionate, 2-(2-hydroxyethyl)-1-methylpropyl 1-piperidine-carboxylate, N,N-diethyl-3-methylbenzamide, dimethyl phthalate, butopyronoxyl, 2,3,4,5-bis(2-butylene)tetrahydro-2-furaldehyde, N,N-diethylcaprylamide, N,N-diethylbenzamide, o-chloro-N,N-diethylbenzamide, dimethyl carbate, di-n-propyl isocinchomeronate, 2-ethylhexane-1,3-diol, N-octylbicycloheptenedicarboximide or piperonyl butoxide;
the organic UV filter is selected from the group consisting of dibenzoylmethane derivative, cinnamic acid derivative, salicylic acid derivative, camphor derivative, triazine derivative, β,β-diphenyl acrylate derivative, p-aminobenzoic acid derivative, polymeric filters and silicone filter;
the inorganic UV filter is selected from the group consisting of coated titanium dioxide, zinc oxide, iron oxide and cerium oxide; and
the active compound is selected from the group consisting of Piroctone Olamine, Tropolone, Hinokitol Selenium Sulfide, Salicylic Acid, Climbazole, Sodium Salicylate, Ciclopiroxolamine, Neem, Basilic oil, Ichtammol, Melaleuca Alternifolia, Centaurea Cyanus, Melia Azadirachta, Farnesol, Sulfur, Clotrimazole, Crotamiton, Zinc Salicylate, Tussilago farfara, Arctium lappa, Zinc Sulfate, Rosmarinus officinalis, Ketoconazole, Myrtrimonium Bromid, Lactic Acid, Chlorohexidine Digluconate, Phenoxyisopropanol, Isopropanol, Glycolic Acid, Tannic acid, Alcohol, Triclosan, Zinc Gluconate, Zinc PCA, Camphor, Aluminium salts, Sodium Lactate, Polyaminopropyl Biguanide, Zinc Acetate, Triethyl Citrate, Ethylhexylglycerol, Aluminium Circonium, Tetrachlorohydrex GLY, Pentetic Acid, Diisopropylamine Aminoethylpropanol, Zinc Ricinoleate, Aluminium Sesquichlorohydrate, Lactic Acid and Triclosan.

9. The composition according to claim 6, wherein the composition is in the form of a solution, suspension, emulsion, paste, ointment, gel, cream, lotion, shampoo, powder, oil, waxe, pencil, shampoo, deodorant-cream, deodorant stick, roll-on, spray, pump spray, aerosol, polish, varnish or hair lacquer.

10. A process for the production of a compound of Formula I according to any one of claims 1 to 5, the process comprising:
converting an ammonium halide of formula II
[NRR¹R²R³]⁺[Hal]⁻ II,
wherein
[Hal]⁻ is Cl^{-, Br}⁻ or I⁻;
R is methoxyethyl, methoxymethyl, ethoxyethyl or ethoxymethyl, and
R¹ to R³ are independantly of each other methyl or ethyl,
to an ammonium hydroxide via ion exchange followed by reacting the ammonium hydroxide with the acid R⁴—COOH,
wherein
R⁴ is linear or branched alkyl with 2 to 4 C atoms, ethyl, propyl, or butyl,
thereby forming a compound of Formula 1.

11. A process for the preparation of a composition according to any one of claim 6-8 or 9, characterised in that at least one compound of Formula I is mixed with a carrier and optionally with further active compounds or auxiliaries.

12. Use of a compound according to any one of claims 1 to 5 as an anti-dandruff agent.

13. Use of a composition according to any one of claim 6-8 or 9 for the treatment of dandruff.

14. A method of treating dandruff comprising administering to a subject in need thereof a compound of Formula I according to any one of claims 1 to 5.

15. A method of treating dandruff comprising administering to a subject in need thereof a composition according to any one of claim 6-8 or 9.
